(19) United States
(12) Patent Application Publication
(10) Pub. No.: US 2007/0021810 A1
(43) Pub. Date: Jan. 25, 2007

(54) Conformable thermal pack apparatus, manufacture and method

(76) Inventor: Kathy Wood Paulin, La Jolla, CA (US)

Correspondence Address: DALINA LAW GROUP, P.C., 7910 IVANHOE AVE. #325, LA JOLLA, CA, 92037, US

(21) Appl. No.: 11/184,514

(22) Filed: Jul. 19, 2005

(51) Int. Cl. A61F 7/00

(52) U.S. Cl. 607/114

(57) ABSTRACT

A therapeutic conformable thermal pack having increased thermal energy storage capacity. The apparatus and method enables therapeutic delivery of more stored heat or cold than previously possible to various areas of a treatment area. A new scientific discovery has been made that shows that ellipsoids and oblate spheroids randomly pack more densely than spheres. The thermal pack comprises thermal retaining elements comprising oblate spheroids and/or ellipsoids allowing embodiments to have an improved thermal density and thermal transfer capability.

### BACKGROUND OF THE INVENTION

1. Field of the Invention

Embodiments of the invention described herein pertain to the field of thermal therapeutic devices and methods. More particularly, but not by way of limitation, one or more embodiments of the invention enable dense storage of thermal energy in a thermal therapeutic pack.

2. Description of the Related Art

There are a number of requirements and/or preferences associated with therapeutic heat/cold application devices. All known devices hold gels, or thermal capacitive devices having a variety of shapes. By heating or cooling the pack and applying the pack to a human or animal the related art devices transfer thermal energy to or from a patient undergoing treatment. The various reasons for applying heat or cold are known, but generally comprise reducing swelling, reducing bruising, or applying heat to increase blood flow to a treatment area for healing purposes. For example, therapeutic devices such as rubber bags exist that filled with hot or cold water and placed on a body part to transfer thermal energy. Filling bags with hot and cold water is inconvenient for someone in need of a thermal pack and many devices were designed and patented to allow for closed devices to be used that did not require application of water. Original packs that used chemicals other than water could only be used as cold packs. Later devices allowed for the application of heat or cold and used non-toxic chemicals and materials. Manufacture of packs that contain gel is a complicated endeavor and other devices were constructed to overcome the limitations of gel based packs. A tear or even small hole in a therapeutic pack that uses a gel as the thermal capacitive element causes a leak in the pack that is messy and renders the device unusable. In addition, gel packs do not conform to body parts well.

U.S. Pat. No. 5,190,033 to Johnson describes a device that is preferably filled with spherical objects that are pea sized up to one half of an inch in diameter. See the Abstract and Col. 2, 11. 63-68. The device consists of a container having an air hole for removing unnecessary air from the device during application. Randomly packed spheres are shown in FIG. 2 showing less than optimal density of packing of thermal components 20. The device is shown in alternate shapes, all of which suffer from the random packing of spheres density problem.

U.S. Pat. No. 5,948,010 to Ademec describes a device that is preferably filled with cracked corn or may be filled with beans, peas, rice, and even sand. See Col. 4, 11. 5-10. The device consists of straps for coupling the device to a human undergoing therapy. The user of beans as thermal capacitive elements provides lower random packing density than even spheres since beans are elongated, flattened and curved. The use of peas for thermal capacitive elements begets the problems of random sphere packing. The use of rice for thermal capacitive elements has a very low random packing density since rice grains are approximately by cylinders that leave large voids when randomly packed. This device may use sand as thermal capacitive elements, however randomly packed sand has a much lower density than spherical particles of equivalent volume since the roughly cube shaped sand particles when randomly packed leave more gaps between sand particles than correspondingly shaped spheres do. Therefore, the device when used with sand has suboptimal density and a tear or hole in the pack of a small size provides problems similar to the gel pack problems wherein a compartment within the pack loses thermal capacitive elements, gives rise to a messy situation and renders a portion or all of the pack ineffective depending on the size of the hole and number of compartments within the pack.

U.S. Pat. No. 6,261,314 to Rich describes a device that is filled with frozen peas. See Col. 2, 11. 22-25. The device consists of hook and loop fasteners for providing shaped therapeutic packs in post-operative situations. Frozen peas are spherical and suffer from the lower random packing problem previously outlined.

U.S. Pat. No. 6,796,996 to Antinoro describes a device that is filled with dried rice, buckwheat hulls, flaxseed, cracked corn, peas or sand. See Col. 1, 11. 66-Col. 2, 11. 1. The device is intended for use with animals. The geometrical shapes of the various thermal capacitive elements all have lower random packing density spheres, except the peas that are spherical. Use of thermal capacitive elements that are spherical yields the problems associated with the random packing of spheres.

U.S. Pat. No. 6,852,121 to Wilson et al., describes a device that is filled water filled capsules that are preferably small, such as the size of peas and substantially spherical, which allows the thermal treatment pack to conform to limbs. See Col. 5, 11. 7-13. Use of thermal capacitive elements that are spherical, such as peas, yields the problems associated with the random packing of spheres.

None of the prior art devices use materials that randomly pack more densely than spheres and for at least the limitations described above there is a need for an improved conformable thermal pack.

### BRIEF SUMMARY OF THE INVENTION

One or more embodiments of the invention enable dense storage of thermal energy in a therapeutic conformable thermal pack. These embodiments enable therapeutic delivery of heat or cold to various areas of a biological treatment area such as a human or animal's body. Use of the embodiments enabled herein is accomplished by heating the apparatus or cooling the apparatus to a desired temperature and applying the apparatus to a treatment area for a human or animal. Applying the apparatus to a treatment area may comprise coupling the apparatus to a treatment area using built-in straps or other coupling techniques or by using a separate device such as a bandage to couple the apparatus to a treatment area, for example wrapping a stretchable bandage around the apparatus and a limb of a patient.

Embodiments of the invention utilize a recent scientific discovery regarding oblate spheroids and ellipsoids. This discovery was recently made by Princeton physicist Paul Chaikin and his collaborators. The oblate spheroid and ellipsoidal geometric shapes differ from spheres in that oblate spheroids are effectively flattened spheres, for example the Earth is not spherical but is an oblate spheroid since the distance from North to South pole is less than the diameter of the Earth at the equator. Similarly ellipsoids are not spherical in nature since they comprise more than one foci from which all points on the surface of the ellipsoid are a fixed distance from the additive distances from the two foci. Both of these geometrical shapes actually randomly pack more densely than spheres of equivalent volume. Before this discovery, scientists did not know that randomly packed shapes could pack more densely than spheres.

Applying this discovery regarding oblate spheroids and ellipsoids to the field of therapeutic thermal packs allows for a more efficient shape to be used for thermal retaining elements residing in a container that is used as a conformable therapeutic pack for example. Using oblate spheroids and/or ellipsoids allows for random packing of these elements that is actually higher than the random packing of equivalent volume spheres. Any size of oblate spheroid and/or ellipsoidal thermal retaining elements may be utilized in embodiments of the invention. Oblate spheroid and ellipsoidal thermal retaining elements may be as small or smaller than a grain of sand or any size larger than a grain of sand depending on the application. Use of thermal retaining elements as opposed to gel allows for a more conformable therapeutic thermal pack. The oblate spheroid or ellipsoidal thermal retaining elements may be solid or hollow and filled with a thermal retaining compound such as gel for example.

Any shape of container may be utilized for retaining the thermal retaining elements. Containers that conform to a particular treatment area contour are in keeping with the spirit of the invention. Containers that are a conformed to a preset shape that may or may not be conformed to other shapes are also in keeping with the spirit of the invention. Embodiments pre-shaped for use on the face, eyes, elbows, wrists, knees, ankles, feet or any other body part are in keeping with the spirit of the invention.

The container may be closed and form an air-tight container or the container may comprise at least one opening smaller than the thermal retaining elements in order to retain the elements within the container. Allowing for the removal of air and retention of thermal retaining elements allows for conforming an embodiment of the invention to a specific body part.

Any manner of attaching the container may be utilized. For example, built in straps or placement of the container under a bandage that is then wrapped around a human or other animal body part is in keeping with the spirit of the invention. Adhesive or non-adhesive embodiments may be constructed or any other method including external coupling may be utilized in retaining an embodiment of the invention to a body part.

The container may be made of material that is absorptive or non-absorptive in nature. The material may also comprise at least one ointment or medicine that is applied to the treatment area. For example, an anticoagulant or antibiotic ointment may be applied to a treatment area via the surface of the container that is also delivering thermal energy such as heat or cold to the same treatment area. Alternatively, or in combination, a cover may be placed over the container that is used in delivering medication or ointments.

Embodiments of the invention may be readily manufactured in a variety of ways so long as the embodiments are capable of retaining the thermal retaining elements. Any material may be used to manufacture the container and the oblate spheroid and/or ellipsoidal thermal retaining elements. For example, materials of various durability or thickness may be utilized for different end requirements, such as military applications that may require rugged construction or materials containing patterns, images or characters printed on the container based on the intended user of the apparatus. Manufacturing embodiments of the invention comprises opening the container, inserting oblate spheroid and/or ellipsoidal thermal retaining elements and closing the container to secure the thermal retaining elements within the container. Stitching, heat bonding or any other method of closing the container to retain the thermal retaining elements is in keeping with the spirit of the invention.

### BRIEF DESCRIPTION OF THE DRAWINGS

The above and other aspects, features and advantages of the invention will be more apparent from the following more particular description thereof, presented in conjunction with the following drawings wherein:

FIG. 1 is a top view of an embodiment of the invention employing ellipsoidal shaped thermal retaining elements.

FIG. 2 is a top view of an embodiment of the invention employing oblate spheroid shaped thermal retaining elements.

FIG. 3 is a top view of an embodiment of the invention employing ellipsoidal and oblate spheroid shaped thermal retaining elements.

FIG. 4 is a top view of an embodiment of the invention employing ellipsoidal and oblate spheroid shaped thermal retaining elements wherein the elements are of varying size.

FIG. 5 shows a flow chart detailing the method of manufacturing the apparatus.

FIG. 6 shows an embodiment designed for facial application.

### DETAILED DESCRIPTION

A therapeutic conformable thermal pack will now be described. In the following exemplary description numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

One or more embodiments of the invention enable dense storage of thermal energy in a therapeutic conformable thermal pack. These embodiments enable therapeutic delivery of heat or cold to various areas of a biological treatment area such as a human or animal's body. Use of the embodiments enabled herein is accomplished by heating the apparatus or cooling the apparatus to a desired temperature and applying the apparatus to a treatment area for a human or animal. Applying the apparatus to a treatment area may comprise coupling the apparatus to a treatment area using built-in straps or other coupling techniques or by using a separate device such as a bandage to couple the apparatus to a treatment area, for example wrapping a stretchable bandage around the apparatus and a limb of a patient.

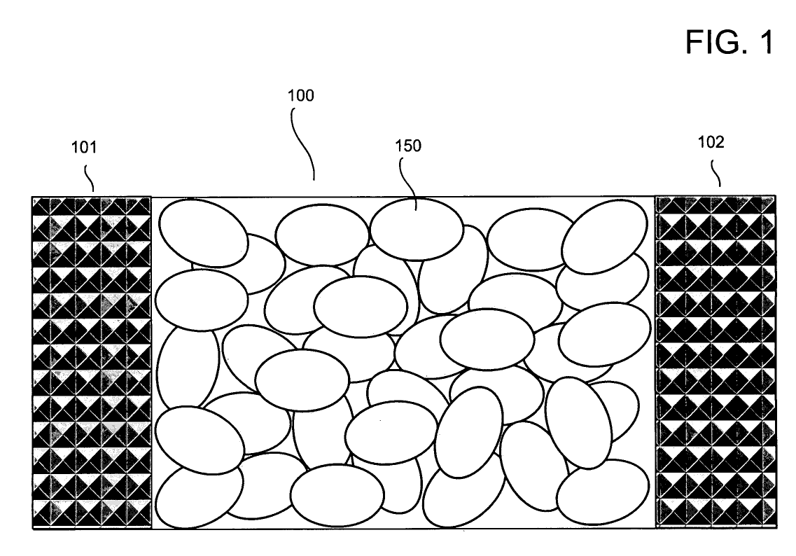

FIG. 1 shows an embodiment of the invention comprising thermal retaining elements that randomly pack more densely than spheres. The thermal retaining elements are shown as relatively large for ease of illustration and one skilled in the art will recognize that any size of thermal retaining elements may be used with embodiments of the invention. The thermal retaining elements may be solid, hollow and may or may not be filled with liquid or gel or any other material so long as the shape of the thermal retaining objects is such that random packing of the objects results in a higher density than packing spheres of similar volume. In this figure, conformable thermal pack 100 comprises optional attachment elements 101 and 102 which may be adhesive or VELCRO couplers for example. Any other manner of attaching the container to a treatment area may be utilized. For example, built in straps or placement of the container under a bandage that is then wrapped around a human or other animal body part is in keeping with the spirit of the invention.

In this embodiment, ellipsoidal thermal retaining elements 150 are held in conformable thermal pack 100 and provide superior random packing with respect to spherical or other shaped thermal retaining elements. Embodiments of the invention are an application of a recent scientific discovery regarding oblate spheroids and ellipsoids. This discovery was recently made by Princeton physicist Paul Chaikin and his collaborators. The oblate spheroid and ellipsoidal geometric shapes differ from spheres in that oblate spheroids are effectively flattened spheres, for example the Earth is not spherical but is an oblate spheroid since the distance from North to South pole is less than the diameter of the Earth at the equator. Similarly ellipsoids are not spherical in nature since they comprise more than one foci from which all points on the surface of the ellipsoid are a fixed distance from the additive distances from the two foci. Both of these geometrical shapes actually randomly pack more densely than spheres of equivalent volume. Before this discovery, scientists did not know that randomly packed shapes could pack more densely than spheres. Applying this discovery regarding oblate spheroids and ellipsoids to the field of therapeutic thermal packs allows for a more efficient shape to be used for thermal retaining elements residing in a container that is used as a conformable therapeutic pack for example. Using oblate spheroids and/or ellipsoids allows for random packing of these elements that is actually higher than the random packing of equivalent volume spheres. Any size of oblate spheroid and/or ellipsoidal thermal retaining elements may be utilized in embodiments of the invention. Oblate spheroid and ellipsoidal thermal retaining elements may be as small or smaller than a grain of sand or any size larger than a grain of sand depending on the application.

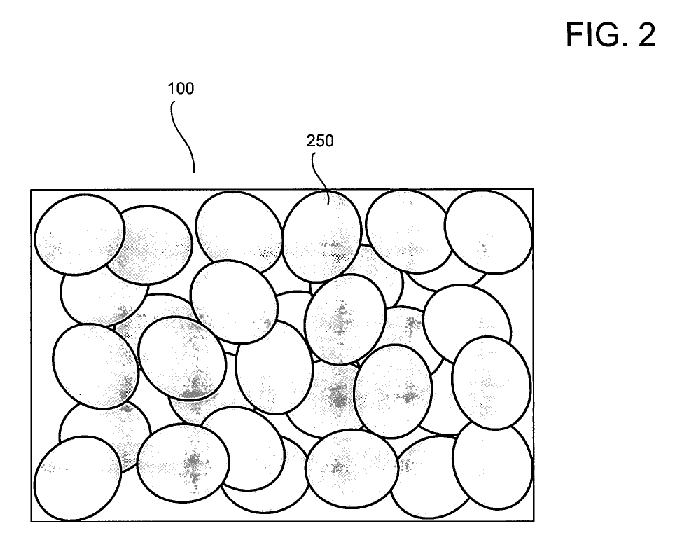

FIG. 2 shows an embodiment of the invention comprising thermal retaining elements that randomly pack more densely than spheres. In this figure, oblate spheroid thermal retaining elements 250 are used in this embodiment of the invention to provide superior random packing with respect to spherical or other shaped thermal retaining elements.

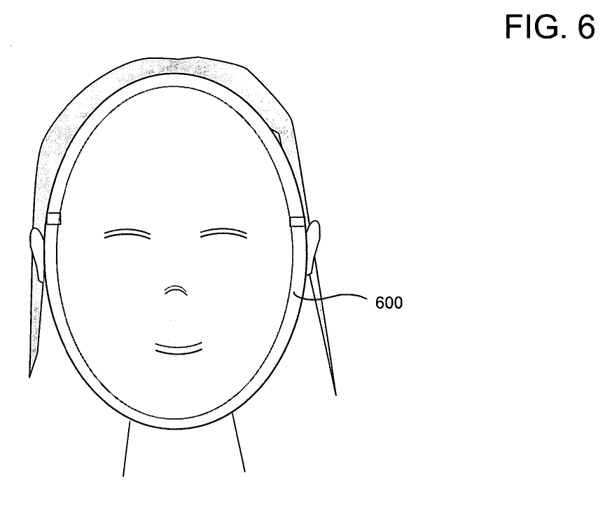

Although FIGS. 1-4, 6 show rectangular shaped containers, any shape of container may be utilized for retaining the thermal retaining elements. Containers that conform to a particular treatment area contour are in keeping with the spirit of the invention. Containers that are a conformed to a preset shape that may or may not be conformed to other shapes are also in keeping with the spirit of the invention. The container may be closed and form an air-tight container or the container may comprise at least one opening smaller than the thermal retaining elements in order to retain the elements within the container. FIG. 2 shows a section of conformable thermal pack 100 allowing for air to escape from the inside of conformable thermal pack 100 when the pack is applied and compressed on a treatment area. Although one small portion of the pack is shown as made from a mesh material, the entire pack may also be constructed using a material with holes that are smaller than any type of thermal retaining element held inside conformable thermal pack 100. For example, the holes in conformable thermal pack 100 at holes 270 are smaller than the thermal retaining elements 250. FIG. 6 shows an embodiment pre-shaped for facial application.

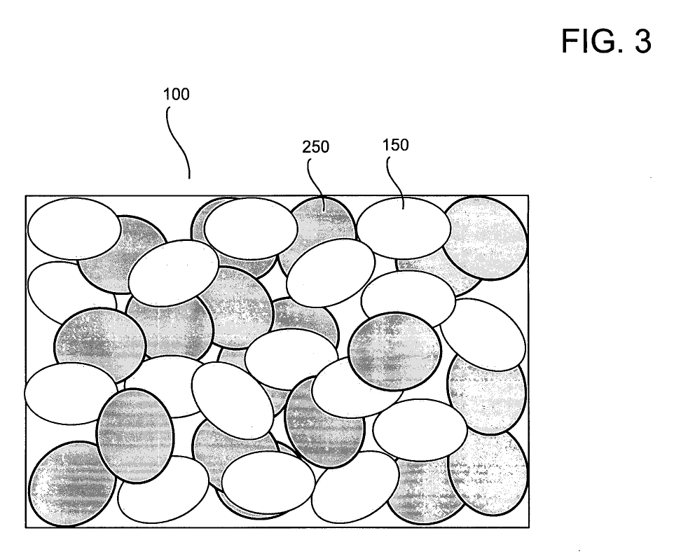

FIG. 3 shows an embodiment of the invention comprising thermal retaining elements that randomly pack more densely than spheres. In this figure, ellipsoidal and oblate spheroid thermal retaining elements 150 and 250 respectively are used in this embodiment of the invention to provide superior random packing with respect to spherical or other shaped thermal retaining elements.

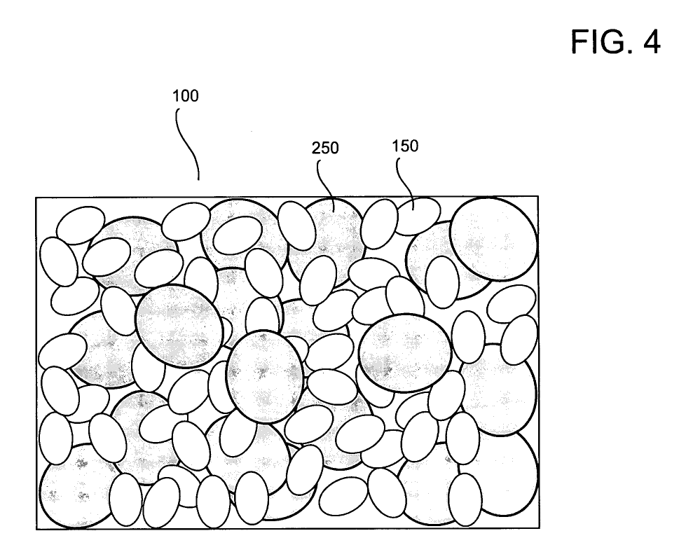

FIG. 4 shows an embodiment of the invention comprising thermal retaining elements that randomly pack more densely than spheres. In this figure, ellipsoidal and oblate spheroid thermal retaining elements 150 and 250 of different sizes are used in this embodiment of the invention to provide superior random packing with respect to spherical or other shaped thermal retaining elements. The sizes of ellipsoidal elements 150 may be different from one another and the sizes of oblate spheroid elements 250 may be different from one another. Alternatively, ellipsoidal elements 150 may be of a given size which may be different from a particular size of oblate spheroid elements 250.

The container shown in FIGS. 1-4, 6 may be made of material that is absorptive or non-absorptive in nature. The material may also comprise at least one ointment or medicine that is applied to the treatment area. For example, an anticoagulant or antibiotic ointment may be applied to a treatment area via the surface of the container that is also delivering thermal energy such as heat or cold to the same treatment area. Alternatively, or in combination, a cover may be placed over the container that is used in delivering medication or ointments.

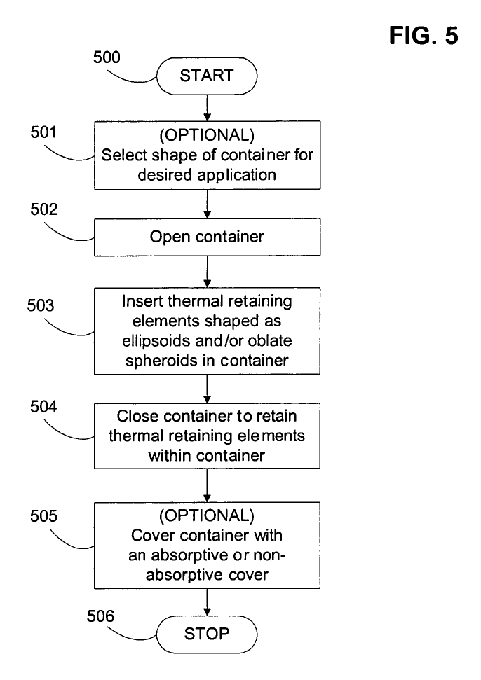

FIG. 5 shows a flow chart detailing a method of manufacturing an embodiment of the invention. The apparatus may be readily manufactured in a variety of ways so long as the embodiments are capable of retaining the thermal retaining elements. Any material may be used to manufacture the container and the oblate spheroid and/or ellipsoidal thermal retaining elements. For example, materials of various durability or thickness may be utilized for different end requirements, such as military applications that may require rugged construction or materials containing patterns, images or characters printed on the container based on the intended user of the apparatus. FIG. 5 shows a flow chart detailing an embodiment of a method for manufacturing the apparatus. Manufacturing embodiments of the invention begins at 500 and comprises the optional step of selecting a container with a particular shape for a particular application. The manufacturing method comprises opening the container at 502, inserting oblate spheroid and/or ellipsoidal thermal retaining elements at 503 and closing the container to secure the thermal retaining elements within the container at 504. Stitching, heat bonding or any other method of closing the container to retain the thermal retaining elements is in keeping with the spirit of the invention. Optionally, the manufacturing method may also comprise covering the container with a cover that is absorptive or non-absorptive (and which may or may not be used in applying medicine or ointments) at 505. The manufacturing process ends at 506.

While the invention herein disclosed has been described by means of specific embodiments and applications thereof, numerous modifications and variations could be made thereto by those skilled in the art without departing from the scope of the invention set forth in the claims.

What is claimed is:

1. A therapeutic conformable thermal pack comprising:
a container configured for physical shape alteration independent of temperature; and,
a multiplicity of thermal retaining elements physically positioned within said container wherein said multiplicity of thermal retaining elements comprise non-spherical components that randomly pack more densely than randomly placed spheres; and,
said container configured to transfer thermal energy in said multiplicity of thermal retaining elements through said container to a treatment area for a biological entity.

2. The therapeutic conformable thermal pack of claim 1 wherein said thermal retaining elements are shaped as oblate spheroids.

3. The therapeutic conformable thermal pack of claim 1 wherein said thermal retaining elements are shaped as ellipsoids.

4. The therapeutic conformable thermal pack of claim 1 wherein said thermal retaining elements are shaped as a combination of oblate spheroids and ellipsoids.

5. The therapeutic conformable thermal pack of claim 1 wherein said thermal retaining elements are not uniform in size.

6. The therapeutic conformable thermal pack of claim 4 wherein said oblate spheroids and ellipsoids are not flexible.

7. The therapeutic conformable thermal pack of claim 1 wherein said container comprises an air-tight surface.

8. The therapeutic conformable thermal pack of claim 1 wherein said container comprises a non-air-tight surface.

9. The therapeutic conformable thermal pack of claim 1 wherein said container comprises an porous surface with pores that are smaller than a thermal retaining element selected from said multiplicity of thermal retaining elements.

10. The therapeutic conformable thermal pack of claim 1 wherein said container comprises a material configured to apply an ointment to said treatment area.

11. The therapeutic conformable thermal pack of claim 1 further comprising:
a cover made from a material configured to apply an ointment to said treatment area

12. The therapeutic conformable thermal pack of claim 1 wherein said container comprises a material selected from the group consisting of absorbent or non-absorbent material.

13. The therapeutic conformable thermal pack of claim 1 configured for application to a face.

14. A method for manufacturing a therapeutic conformable thermal pack comprising:
opening a container configured for physical shape alteration independent of temperature;
inserting a multiplicity of thermal retaining elements into said container wherein said multiplicity of thermal retaining elements comprise non-spherical components that randomly pack more densely than randomly placed spheres; and,
closing said container to retain said multiplicity of thermal retaining elements.

15. The method of claim 14 wherein said inserting comprises inserting non-spherical components that are shaped as oblate spheroids.

16. The method of claim 14 wherein said inserting comprises inserting non-spherical components that are shaped as ellipsoids.

17. The method of claim 14 wherein said inserting comprises inserting non-spherical components that are shaped as oblate spheroids and ellipsoids.

18. The method of claim 14 wherein said inserting comprises inserting non-spherical components that are not uniform in size.

19. The method of claim 14 wherein said container is configured to apply medication.

20. The method of claim 14 further comprising:
covering said container with a cover configured to apply medication.
